(19) United States
(12) Patent Application Publication
(10) Pub. No.: US 2024/0115255 A1
(43) Pub. Date: Apr. 11, 2024

(54) SURGICAL ANCHORS, INSTRUMENTATION, AND METHODS OF USE

(71) Applicant: INTERNATIONAL LIFE SCIENCES, LLC d/b/a ARTELON, Marietta, GA (US)

(72) Inventors: L. Chase THORNBURG, Cumming, GA (US); Justin C. SLUDER, Dallas, GA (US); Jantzen COLE, Marietta, GA (US)

(21) Appl. No.: 18/481,223

(22) Filed: Oct. 4, 2023

Related U.S. Application Data
(60) Provisional application No. 63/378,553, filed on Oct. 6, 2022.

(51) Int. Cl. A61B 17/04

(52) U.S. Cl. CPC A61B 17/0401; A61B 2017/0409; A61B 2560/02

(57) ABSTRACT

Surgical anchors, instrumentation, and methods. In one example, a surgical anchor includes an anchor body, an elastic member, and an elongated, flexible member. The surgical anchor is configured such that exerting a pull force on the elongated flexible member in a direction substantially aligned an axis of the anchor body imparts a force on the anchor body that includes a rotational force component that is transverse to the anchor body axis.

### CROSS-REFERENCE TO RELATED APPLICATIONS

This patent application claims the benefit of and priority to U.S. provisional patent application Ser. No. 63/378,553 filed Oct. 6, 2022 for “SURGICAL ANCHORS, INSTRUMENTATION, AND METHODS OF USE” the entire contents of which are hereby incorporated by this reference.

### RELATED FIELDS

Surgical anchors, instrumentation, and methods of use.

### BACKGROUND

Surgical anchors, instrumentation, and methods of use are known for anchoring flexible members and other constructs in the bony anatomy. This patent describes improved surgical anchors, instruments, and methods of use that provide several benefits over earlier anchoring systems and methods.

### SUMMARY

In one exemplary illustration, a surgical anchor may include an anchor body, an elastic member, and an elongated flexible member. The anchor body may include an anchor body axis. The elastic member may be associated with the anchor body. The elongated flexible member maybe associated with the elastic member and extend away from the elastic member and the anchor body. The surgical anchor may be configured such that exerting a pull force on the elongated flexible member in a direction substantially aligned with the anchor body axis imparts a force on the anchor body that includes a rotational force component that is transverse to the anchor body axis.

In another exemplary illustration, a surgical anchor may include an anchor body and an elongated flexible member. The anchor body may include an anchor body axis. The elongated flexible member may be associated with and extend away from the anchor body. The surgical anchor may be configured such that exerting a pull force on the elongated flexible member in a direction substantially aligned with the anchor body axis imparts a force on the anchor body that includes a rotational force component that is transverse to the anchor body axis.

### BRIEF DESCRIPTION OF FIGURES

FIG. 1 shows an example of a surgical anchor.

FIG. 2 shows the elastic member and elongated flexible member of the FIG. 1 surgical anchor.

FIGS. 3a-h show an example of an anchor body of a surgical anchor from several viewpoints.

FIGS. 4a-g show an example of a method of securing a surgical anchor in bony anatomy.

FIG. 5 shows examples of an elastic member.

### DETAILED DESCRIPTION

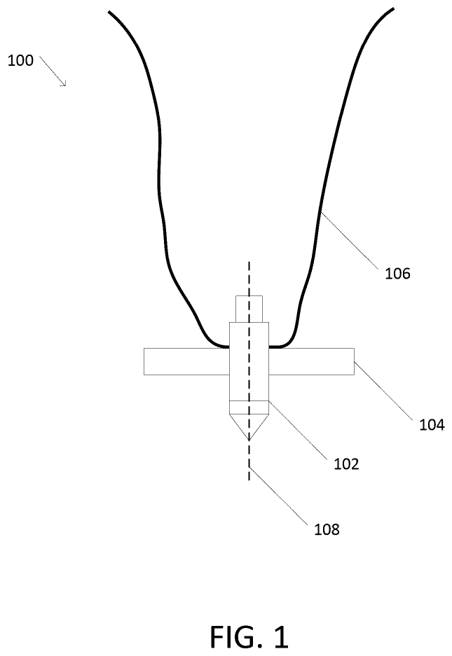

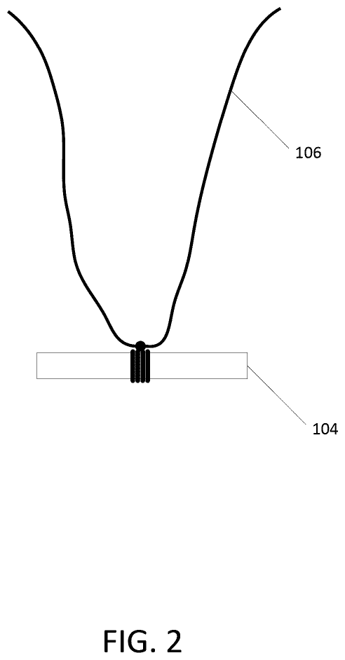

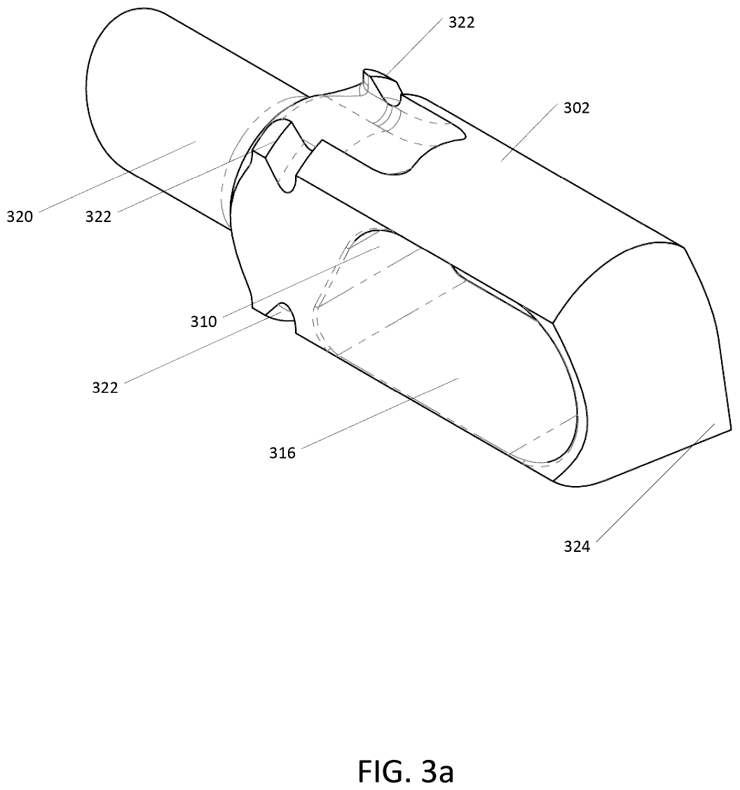

### Example Surgical Anchor—FIGS. 1, 2

FIG. 1 shows an example of a surgical anchor 100. The surgical anchor 100 includes an anchor body 102, an elastic member 104, and an elongated flexible member 106. In this example, the elastic member 104 is associated with the anchor body 102 and the elongated flexible member 106 is associated with the elastic member 104 and extends away from the elastic member 104 and the anchor body 102. The anchor body 102 has an anchor body axis 108 (more particularly in this example a longitudinal anchor body axis).

The anchor body 102 may be a rigid anchor body, such as one made of polyetheretherketone (PEEK) or another thermoplastic material. The elastic member 104 may be an elastic mesh such as an Artelon® FlexBand® or other flexible, elastic strip of mesh material such as described in further detail below. The elongated flexible member 106 may be a suture or other material thread.

FIG. 2 shows the elastic member 104 and elongated flexible member 106 of the surgical anchor 100 of FIG. 1 without the anchor body. As shown in FIG. 2, the elongated flexible member 106 is fixed to the elastic member 104 in a non-sliding fashion. In this particular example, the elongated flexible member 106 is tied around a central portion of the elastic member 104 by wrapping the member 106 four consecutive times around the elastic member 104 and then tying the two tails of the elongated member 106 over the top of the wraps to secure them. In this particular example, the elongated flexible member 106 is not pierced through the elastic member 104.

The surgical anchor 100 of FIGS. 1 and 2 is configured such that exerting a pull force on the elongated flexible member 106 in a direction substantially aligned with the anchor body axis 108 (e.g., in a direction that is parallel to or within 0 degrees to 30 degrees to the anchor body axis) imparts a force on the anchor body 102 that includes a rotation force component that is transverse to the anchor body axis 108. As described in further detail below, this may result of rotation of the anchor body 102 in a bone hole, facilitating anchoring of the surgical anchor 100 in the bone hole.

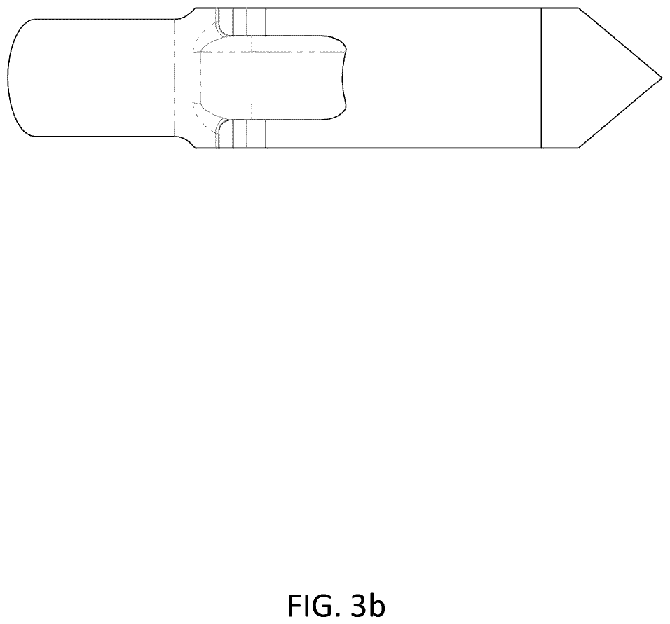

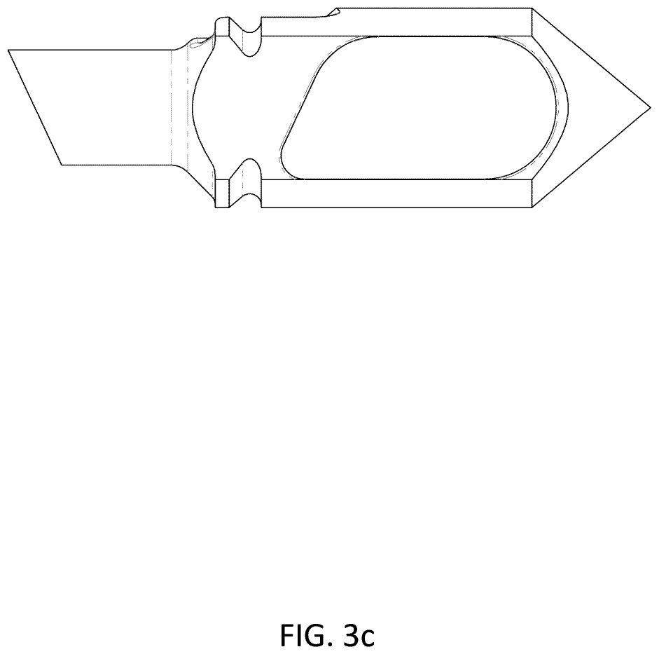

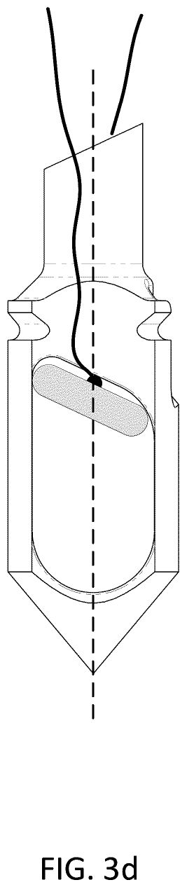

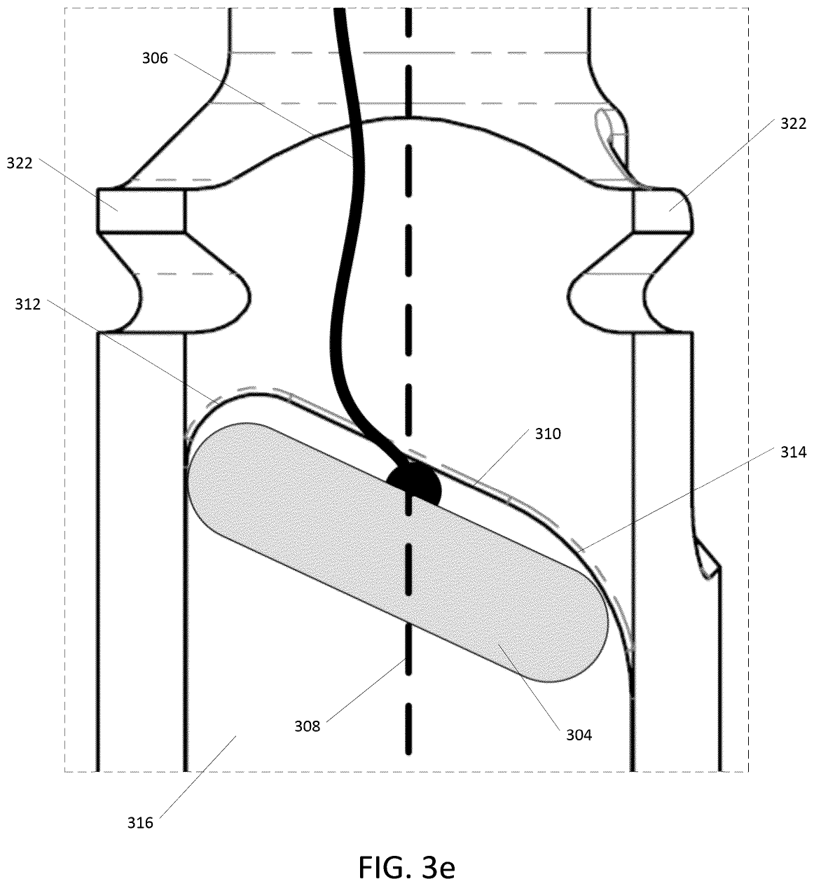

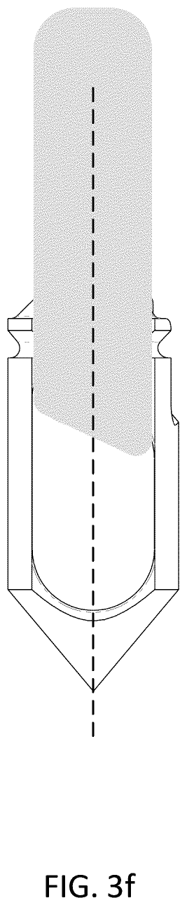

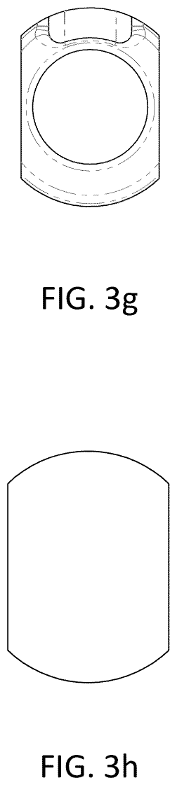

### Example Anchor Body—FIGS. 3a-h

FIGS. 3a-h show another example of an anchor body 302. In this example, the anchor body 302 includes a slanted surface 310 that is slanted relative to the anchor body axis 308 such that exerting the pull force described above on the elongated flexible member imparts a rotational force component through interaction of at least one of the elastic member and the elongated flexible member with the slanted surface 310.

As shown best in FIGS. 3d and 3e, the slated surface 310 is asymmetric relative to the anchor body axis 308. For instance, as shown in FIGS. 3d and 3e, to the left of the anchor body axis 308 the slanted surface 310 slopes generally upward and to the right of the anchor body axis 308 the slanted surface 310 slopes generally downward. The slanted surface 310 slopes between a proximal end 312 and a distal end 314.

In the particular example shown in the figures, the slanted surface 310 slopes at an angle of approximately 65 degrees relative to the longitudinal axis of the anchor body 302. In other examples, the slanted surface may slope at an angle of approximately 60-70 degrees relative to the longitudinal axis, or approximately 50-80 degrees relative to the longitudinal axis.

In the particular example shown in FIGS. 3a-h, the slanted surface 310 is a surface of an eyelet 316 extending through the anchor body 302. As shown in FIGS. 3d-f, the elastic member 304 and the elongated flexible member 306 extend through the eyelet 316. FIGS. 3d and 3e show in partial cross-section how portions of the elastic member 304 and elongated flexible member 306 abut the slanted surface 310 of the eyelet 316. As shown in FIG. 3d, the elongated flexible member 306 is wrapped around the slanted surface 310, with the two tails of the elongated flexible member 306 extending proximally away from the anchor body 302.

The anchor body 302 shown in FIGS. 3a-h includes several bone engagement, features that facilitate anchoring when the anchor body 302 is rotated.

One of these features is proximal bone engagement feature 318, which is located on the same side of the anchor body 302 as the distal end 314 of the slanted surface 310. The surgical anchor is configured such that exerting a pull force on the elongated flexible member 306 with rotate the proximal bone engagement feature 318 to an engagement position, which is described in further detail below. The proximal bone engagement feature 318 is located on a proximal end of a post 320 of the anchor body 302. The post 320 is configured to connect the anchor body 302 to an insertion instrument. The proximal end of the post 320 is sloped in an opposite direction to the slanted surface 310 (e.g. such that the proximal bone engagement feature 318 is at the proximal-most part of the post 320), which facilitates the functionality of the proximal bone engagement feature 318.

In some implementations, the slope of the proximal end of the post 320, including the proximal bone engagement feature 318, may be configured to facilitate rotation of the anchor body 302 when the elongated flexible member is pulled. For instance, as the elongated flexible member is pulled, the proximal bone engagement feature 318 may engage the wall of the bone opening, and the sloped surface of the proximal end may provide for and facilitate rotation of the anchor body 302.

Another one of the bone engagement features of the anchor body 302 are the circumferential engagement features 322 extending around a portion of the anchor body 302. The surgical anchor is configured such that externing a pull force on the elongated flexible member will also rotate these circumferential engagement features 322 to an engagement position, which is described in further detail below.

Another one of the bone engagement features of the anchor body 302 is the distal engagement feature 324 at a distal end of the anchor body 302. In this example, the distal engagement feature 324 is a tapered tip of the anchor body 302, which also facilitates locating the anchor body 302 in a formed hole in the bony anatomy. The surgical anchor is configured such that externing a pull force on the elongated flexible member will also rotate the distal engagement feature 324 to an engagement position, which is described in further detail below.

As shown in FIG. 3a, the sides of the anchor body with the openings of the eyelet may include flattened or recessed areas to provide clearance for the elastic member.

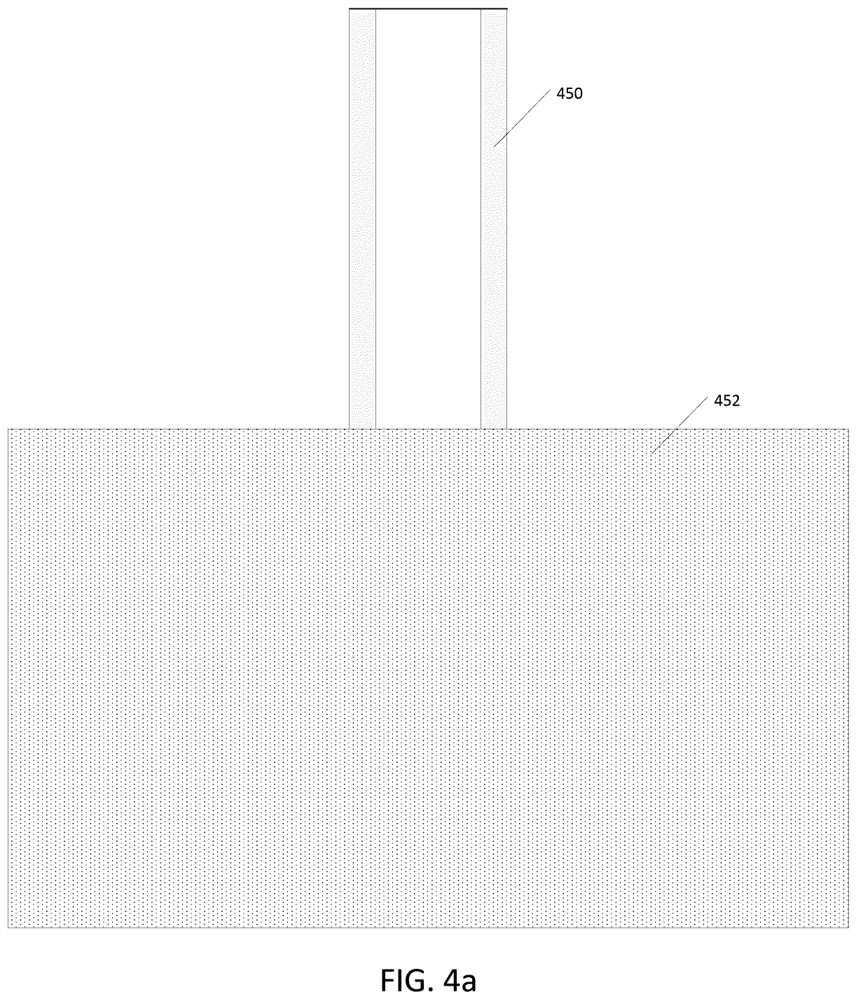

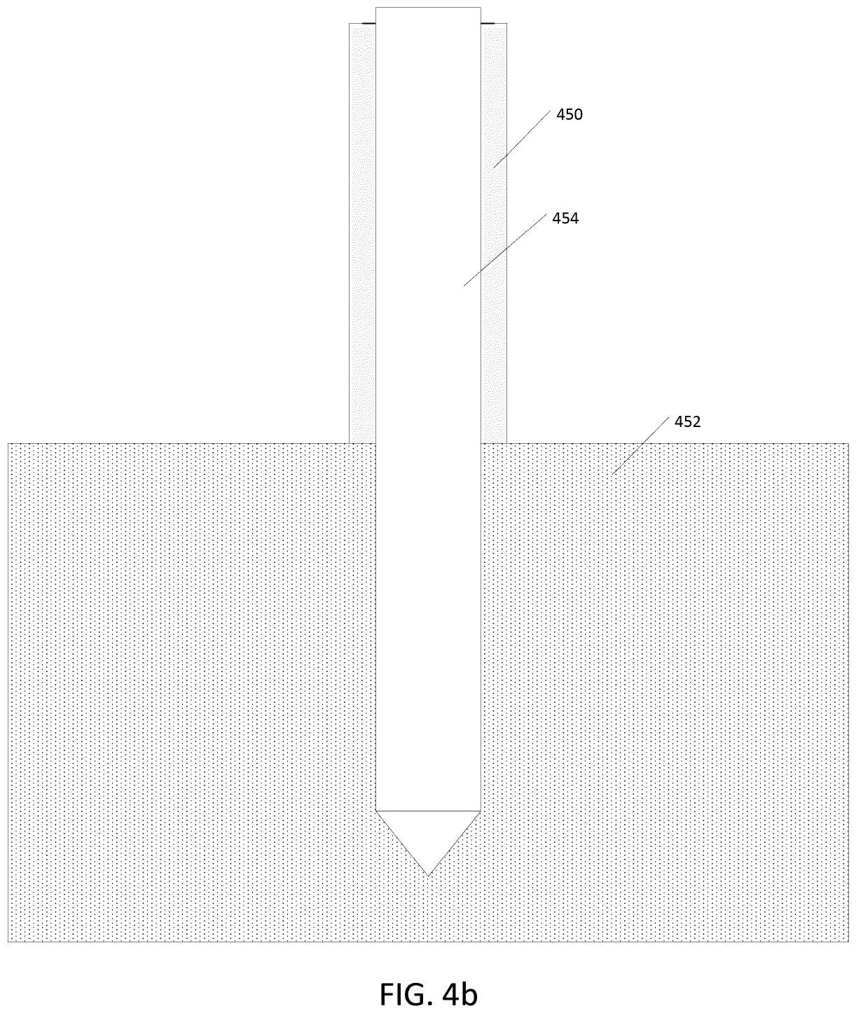

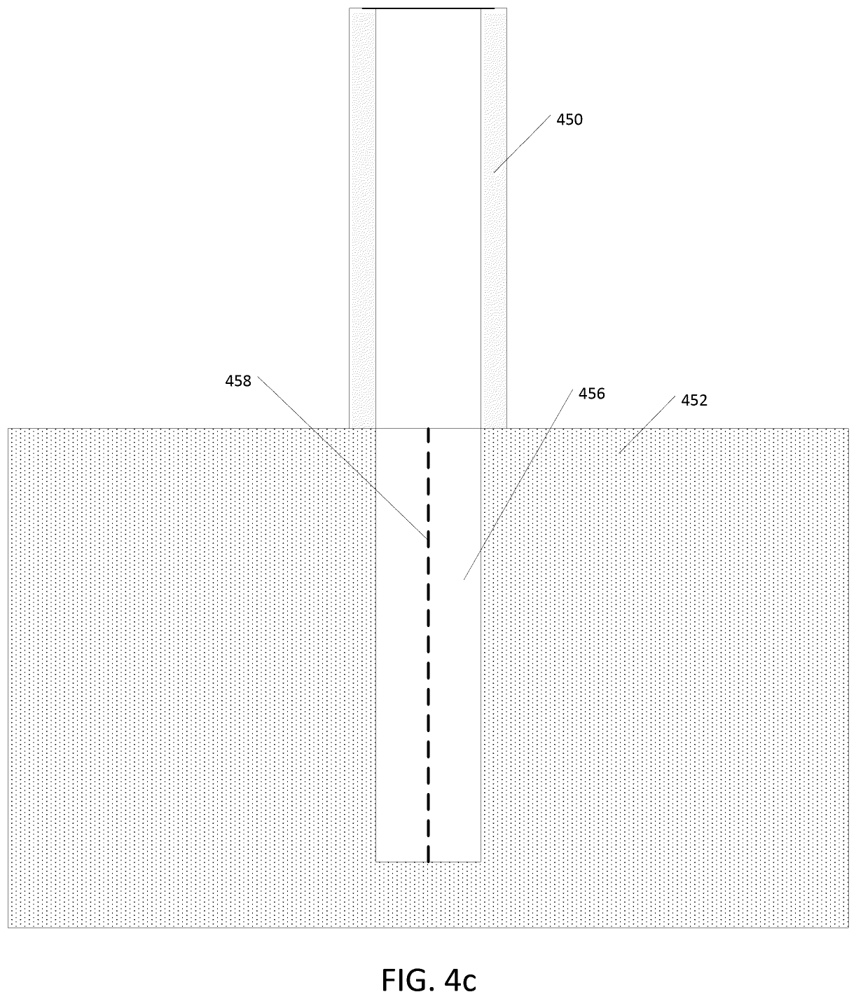

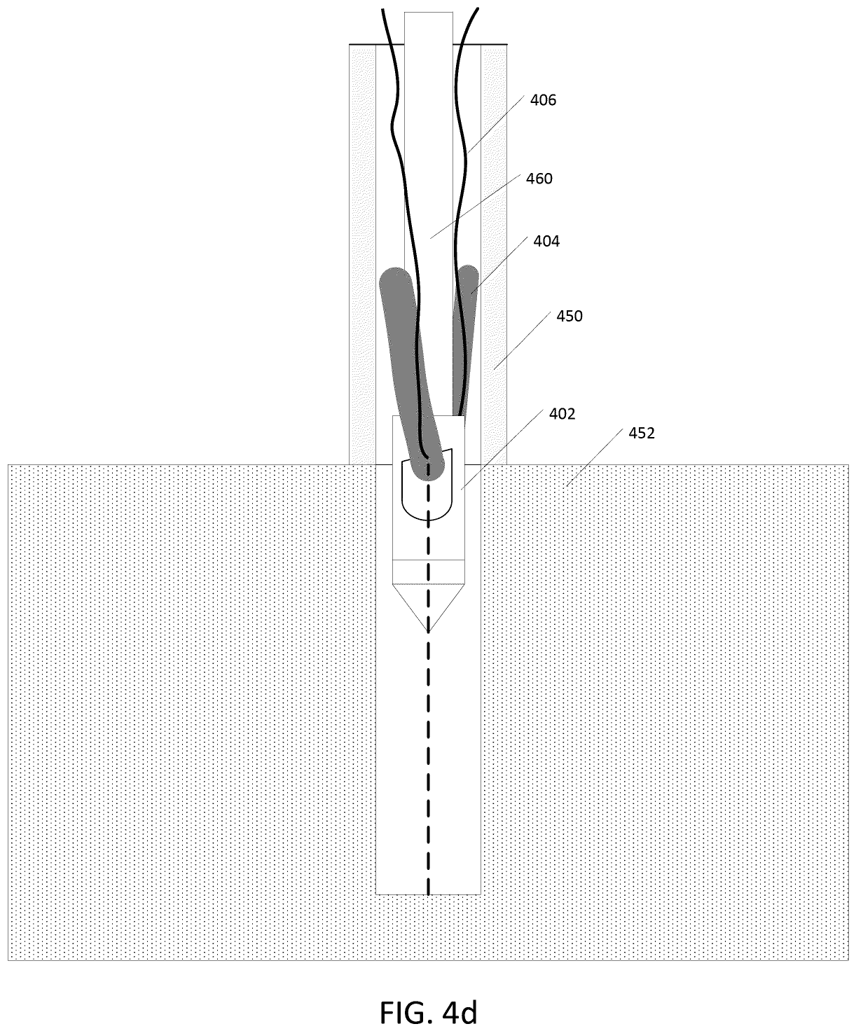

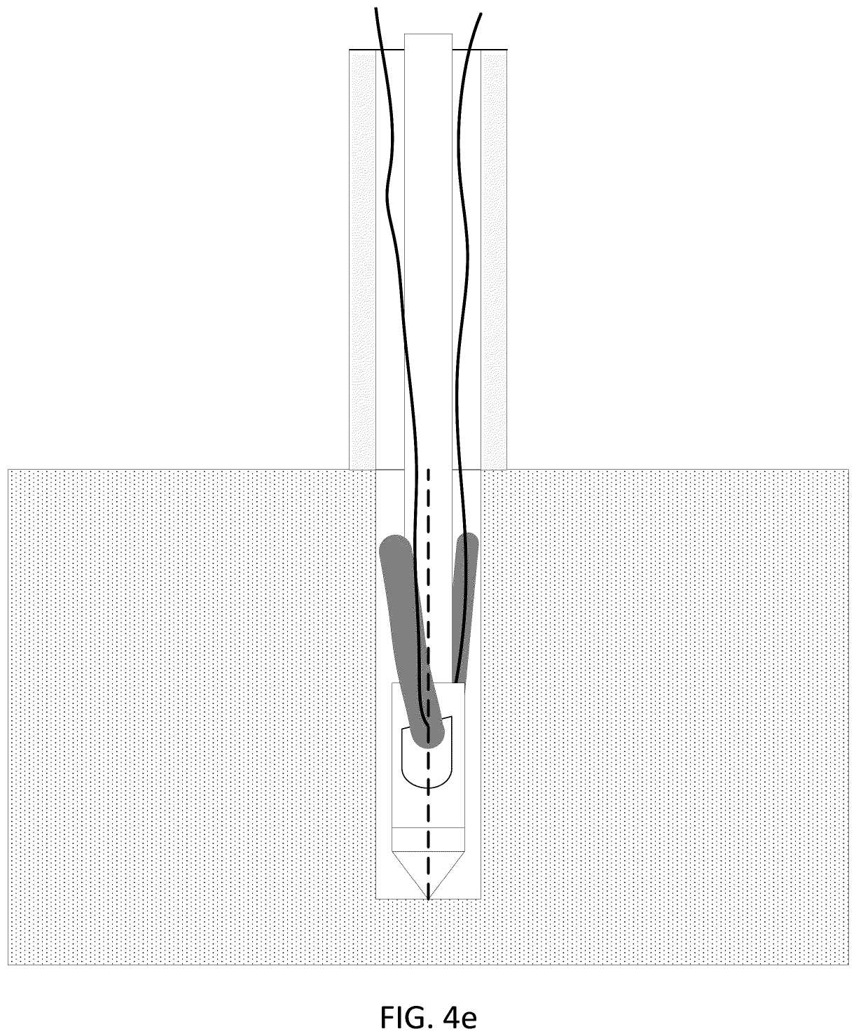

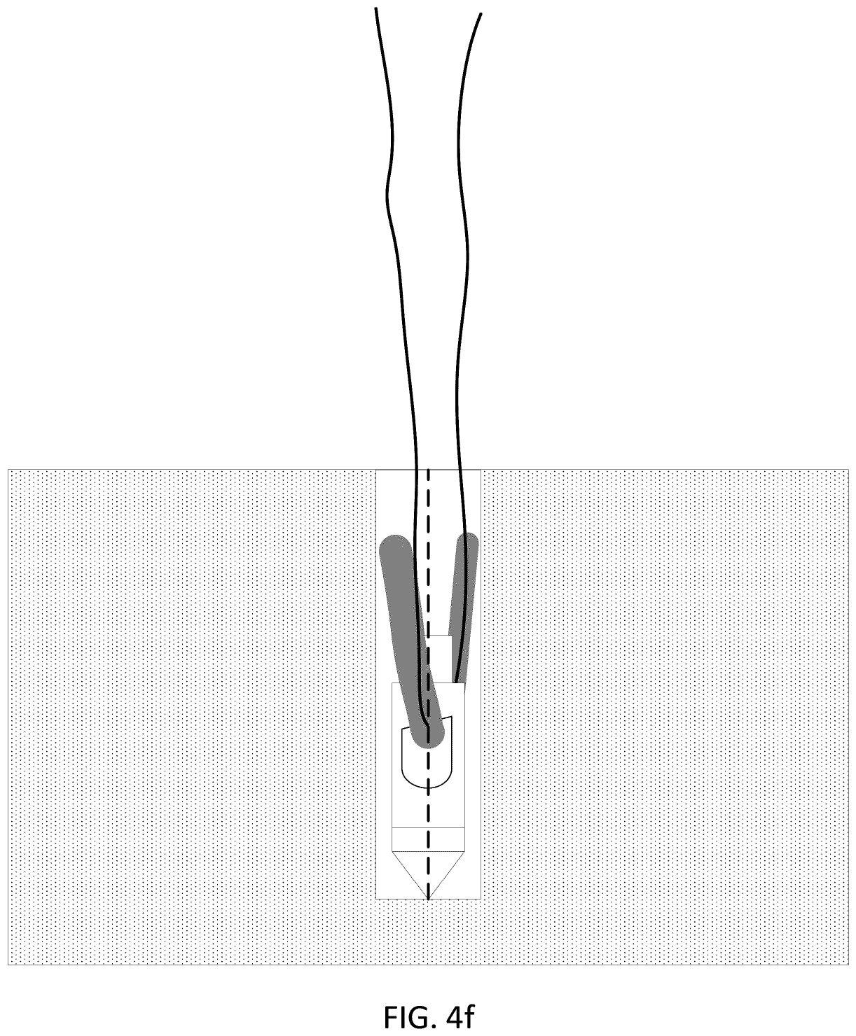

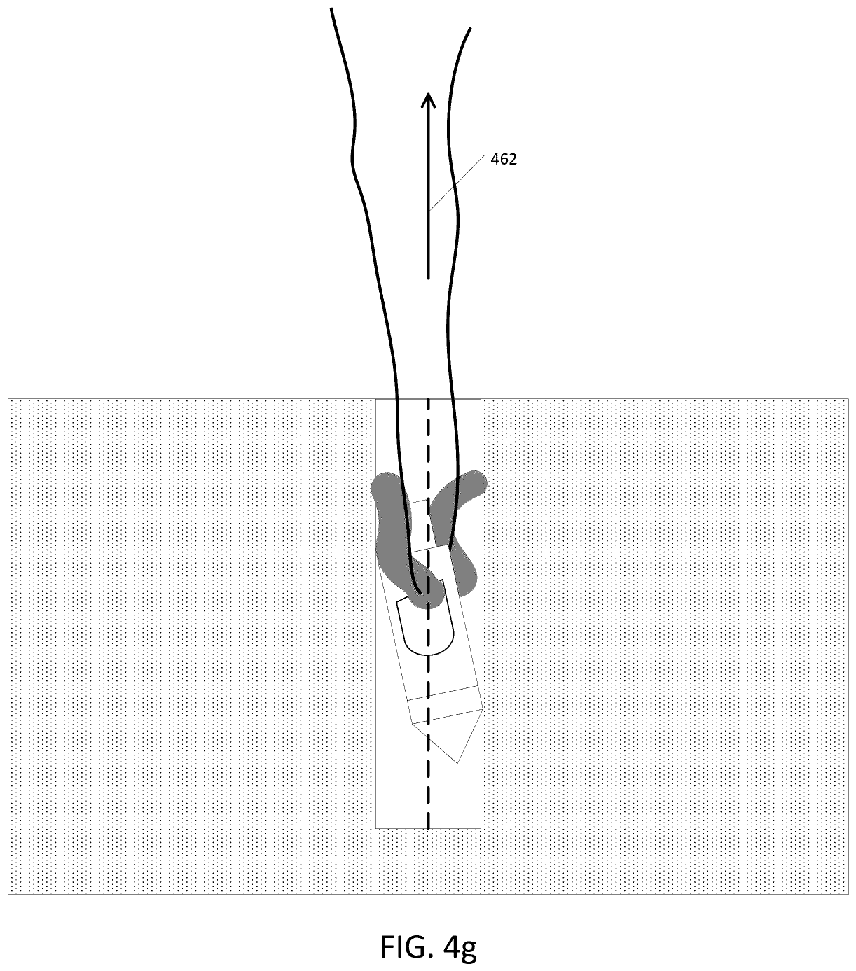

### Example Method—FIGS. 4a-g

FIGS. 4a-g illustrate one example method of implanting a surgical anchor, such as any of the above described surgical anchors.

In FIG. 4a, a guide 450 (e.g. a drill guide) is positioned relative to the bony anatomy 452.

In FIG. 4b, a cutter 454 (e.g. a guide pin with a cutting surface at its distal end) is passed through guide 450 and used to form an opening (e.g. a blind hole or a through tunnel) in the bony anatomy 452.

FIG. 4c shows an opening 456 formed in the bony anatomy 452, the opening including a longitudinal axis 458.

FIG. 4d shows a surgical anchor 400 mounted on a distal end of a surgical instrument 460 and being guided into the opening 456 using guide 450.

FIG. 4e shows the surgical anchor 400 positioned at the bottom of the opening 456.

FIG. 4f shows the drill guide 450 and instrument 460 removed, leaving two tails of the elongated flexible member 406 extending out of the opening 456.

In FIG. 4g a pull force 462 is exerted on the elongated flexible member in a direction substantially aligned with the anchor body axis and the opening longitudinal axis 458 (e.g. within 30 degrees of the axis) to impart a force on the anchor body 402 that includes a rotational force component that is transverse to the axis 458 such that the anchor body 402 rotates in the opening 458 and engaging or further engaging one or more of the proximal, circumferential, and distal engagement features of the anchor body 402 with the wall of the opening, thereby anchoring the surgical anchor in the opening. As can also be seen in FIG. 4g, the anchor body 402 will migrate proximally in the opening 458 in response to the force.

In the example shown in the figures, the elastic member is relatively short and is entirely located in the bone opening after anchoring. In other example, the elastic member may be longer and one or two tails of the elastic member may extend out of the bone opening after anchoring, such as for securing to soft tissue to be repaired.

In this example, subsequently, the now-anchored elongated flexible member may be used to complete a surgical procedure (e.g. secure an implant and/or soft tissue to the bony anatomy in which it is anchored). Although not shown, the ends of the elongated flexible member may each include a surgical needle to facilitate securing the elongated flexible member to an implant and/or soft tissue.

In some non-limiting examples, the opening formed in the bony anatomy may have a depth of 10-30 mm (or 15-25 mm, or 17-19 mm), the final anchored depth of the anchor body may be approximately 2-8 mm from the bottom of the opening. In some non-limiting examples, the diameter of the opening may be the same size as or slightly smaller than the anchor body, facilitating a press-fit insertion of the surgical anchor.

### Example Elastic Member

As noted earlier, the surgical anchors, instrumentation, and described above may be used in conjunction with an elastic member. In some embodiments, the elastic member may be an Artelon® FlexBand.® The elastic member may be an elongated, flexible, elastic strip of material. The elastomeric member may be a degradable biomaterial matrix woven from wet-spun fibers of polycaprolactone based-polyurethane urea (PUUR) that have been knitted into textile strips for optimal mechanical properties and ease of use. The clinical efficacy of the elastomeric member may be generated from the combination of the chemical composition, fiber spinning, and the textile manufacturing process.

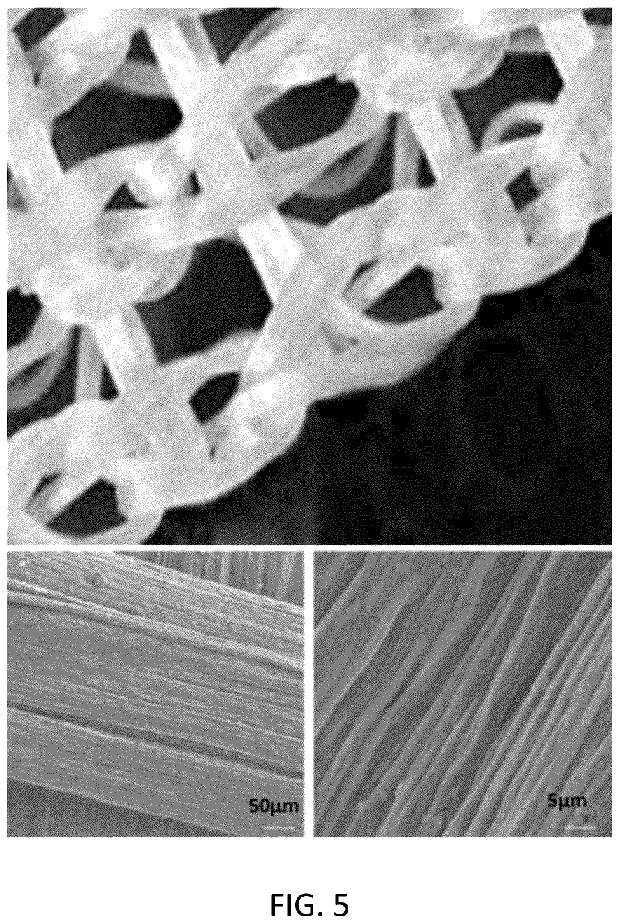

FIG. 5 shows magnified views of one example of such an elastic member, showing the knitted fibers and pores of the matrix. In some embodiments, the matrix has a porosity range of approximately 8 um-600 um, with the majority of the pores concentrated in the smaller subrange. The porous nature of the elastic member provides a “roughened” surface, helping to grip the bone and provide friction in the bone opening. The compressibility of the elastic member also helps to fill voids between the anchor body and the bone.

### CONCLUSION

The foregoing is provided by way of example only. Additions, deletions, substitutions, modifications, and other changes may be made to the surgical anchors, instrumentation, and methods described above without departing from the scope or spirit of our inventions.

1. A surgical anchor, comprising:
an anchor body, the anchor body comprising an anchor body axis; and
an elongated flexible member extending away from the anchor body;
wherein the surgical anchor is configured such that exerting a pull force on the elongated flexible member in a direction substantially aligned with the anchor body axis imparts a force on the anchor body that includes a rotational force component that is transverse to the anchor body axis.

2. The surgical anchor of claim 1, further comprising an elastic member associated with the anchor body, the elongated flexible member associated with the elastic member.

3. The surgical anchor of claim 2, wherein the anchor body comprises a slanted surface that is slanted relative to the anchor body axis, wherein exerting the pull force on the elongated flexible member imparts the force on the anchor body including the rotational force component through interaction of at least one of the elastic member and the elongated flexible member with the slanted surface.

4. The surgical anchor of claim 3, wherein the slanted surface is asymmetric relative to the anchor body axis.

5. The surgical anchor system of claim 4, wherein the anchor body axis is a longitudinal anchor body axis.

6. The surgical anchor of claim 3, wherein the slanted surface is a surface of an eyelet extending through the anchor body.

7. The surgical anchor of claim 6, wherein the elastic member and the elongated flexible member extend through the eyelet.

8. The surgical anchor of claim 7, wherein the elongated flexible member wraps around the slanted surface.

9. The surgical anchor of claim 3, wherein the slanted surface extends between a distal end and a proximal end, wherein the anchor body further comprises at least one proximal bone engagement feature located on the same side of the anchor body as the distal end of the slanted surface.

10. The surgical anchor of claim 9, wherein the surgical anchor is configured such that exerting the pull force on the elongated flexible member rotates the proximal bone engagement feature to an engagement position.

11. The surgical anchor of claim 9, wherein the anchor body further comprises a post configured to connect the anchor body to an insertion instrument, wherein the proximal bone engagement feature is located on a proximal end of the post.

12. The surgical anchor of claim 11, wherein the proximal end of the post is sloped in an opposite direction to the slanted surface.

13. The surgical anchor of claim 3, wherein the anchor body further comprises a circumferential engagement feature extending around at least a portion of the anchor body, wherein the surgical anchor is configured such that exerting the pull force on the elongated flexible member rotates the circumferential engagement feature to an engagement position.

14. The surgical anchor of claim 3, wherein the anchor body further comprises a distal engagement feature at a distal end of the anchor body, wherein the surgical anchor is configured such that exerting the pull force on the elongated flexible member rotates the distal engagement feature to an engagement position.

15. The surgical anchor of claim 2, wherein the elongated flexible member is fixed to the elastic member in a non-sliding fashion.

16. The surgical anchor of claim 15, wherein the elongated flexible member is tied around a central portion of the elastic member.

17. The surgical anchor of claim 2, wherein the elastic member is an elastic mesh.

18. The surgical anchor of claim 17, wherein the elastic mesh is a matrix of knitted fibers with a porosity range of approximately 8 um-600 um.

19. The surgical anchor of claim 1, wherein the anchor body comprises a slanted surface that is slanted relative to the anchor body axis, wherein exerting the pull force on the elongated flexible member imparts the force on the anchor body including the rotational force component through interaction of the elongated flexible member with the slanted surface.

20. The surgical anchor of claim 1, wherein the anchor body further comprises a proximal bone engagement on a sloped proximal end of the anchor body.

21. The surgical anchor of claim 1, wherein the anchor body comprises an eyelet including slanted surface that is slanted relative to the anchor body axis, wherein the anchor body further comprises a proximal bone engagement, on a sloped proximal end of the anchor body; wherein the sloped proximal end is sloped in an opposite direction to the slanted surface.
